(19) United States
(12) Patent Application Publication
(10) Pub. No.: US 2002/0031810 A1
(43) Pub. Date: Mar. 14, 2002

(54) Nucleotide sequences coding for the lipA gene

(75) Inventors: Bettina Moeckel, Duesseldorf (DE); Walter Pfefferle, Halle (DE); Michael Buchholz, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/940,486

(22) Filed: Aug. 29, 2001

(30) Foreign Application Priority Data
Aug. 31, 2000 (DE) 100 42 742.1

(51) Int. Cl. C12P 13/08; C12N 9/02; C07H 21/04; C12N 1/21

(52) U.S. Cl. 435/117; 435/189; 435/320.1; 435/252.3; 536/23.2

(57) ABSTRACT

Nucleotide sequences from coryneform bacteria coding for the lipA gene and a process for fermentative preparation of amino acids by attenuation of the lipA gene.

### BACKGROUND OF THE INVENTION

1. Field of the Invention

The invention relates to nucleotide sequences from coryneform bacteria coding for the lipA gene and a process for fermentative preparation of amino acids, in particular L-lysine, by attenuation of the lipA gene. The lipA gene codes for lipoic acid synthetase.

2. Description of the Background

L-amino acids, in particular L-lysine, are used in human medicine and in the pharmaceutical industry, in the foodstuffs industry and in particular in animal nutrition.

It is known that amino acids can be prepared by fermentation by strains of coryneform bacteria, in particular Corynebacterium glutamicum. Due to its great importance, work is constantly aimed at improving the method of preparation. Process improvements may relate to fermentation-engineering measures such as, for example, stirring and supplying with oxygen, or the composition of the culture media such as, for example, the sugar concentration during fermentation, or working up of the product form by, for example, ion exchange chromatography or the intrinsic performance characteristics of the microorganism itself.

In order to improve the performance characteristics of these microorganisms, the methods of mutagenesis, selection, and mutant choice are applied. In this way, strains are obtained which are resistant to antimetabolites or auxotrophic for regulatorily important metabolites and which produce amino acids. For some years now, the methods of recombinant DNA engineering have also been used for the strain improvement of L-amino acid-producing strains of Corynebacterium. However, there still remains a need for improved microorganisms for producing amino acids.

### SUMMARY OF THE INVENTION

It is an object of the present invention to provide nucleotides which are usefull for the production of amino acids.

It is another object of the present invention to provide new methods for the improved fermentative preparation of amino acids, in particular L-lysine.

Thus, the invention provides a polynucleotide isolated from coryneform bacteria, containing a polynucleotide sequence coding for the lipA gene, chosen from the group

(a) a polynucleotide which is at least 70% identical to a polynucleotide which codes for a polypeptide which contains the amino acid sequence of SEQ ID No. 2,

(b) a polynucleotide which codes for a polypeptide which contains an amino acid sequence which is at least 70% identical to the amino acid sequence of SEQ ID No. 2,

(c) a polynucleotide which is complementary to the polynucleotides under (a) or (b), and

(d) a polynucleotide containing a sequence of at least 15 nucleotides from the polynucleotide sequence (a), (b), or (c), where the polypeptide preferably has the activity of lipoic acid synthetase.

The invention also provides the polynucleotide mentioned above, which is preferably a DNA capable of replication containing:

(i) the nucleotide sequence shown in SEQ ID No. 1, or

(ii) at least one sequence which corresponds to sequence (i) within the region of degeneration of the genetic code, or

(iii) at least one sequence which hybridizes with the sequences which are complementary to sequence (i) or (ii), and, optionally,

(iv) functionally neutral sense mutations in sequence (i).

The invention additionally provides:

a polynucleotide capable of replication, in particular DNA, containing the nucleotide sequence shown in SEQ ID No. 1;

a polynucleotide which codes for a polypeptide which contains the amino acid sequence shown in SEQ ID No. 2;

a vector containing parts of the polynucleotide according to the invention with, however, a sequence of at least 15 of the nucleotides in the claimed sequence

and coryneform bacteria in which the lipA gene is attenuated, in particular by an insertion or deletion.

In addition, the present invention provides a process for preparing an L-amino acid, comprising:

(a) fermenting a coryneform bacteria which produces the L-amino acid and in which at least the lipA gene is attenuated,

(b) enriching the L-amino acid in the medium or in the cells of the bacteria, and (c) isolating the L-amino acid.

The invention also provides polynucleotides which substantially consist of a polynucleotide sequence which are obtainable by screening by means of hybridization of an appropriate gene library from a coryneform bacterium which contains the complete gene or parts thereof, with a probe which contains the sequence for the polynucleotide according to the invention or a fragment thereof and isolating the polynucleotide sequence.

Thus, the present invention also provides a process for identifying RNA, cDNA, or DNA which code for lipoic acid synthetase or exhibit a high similarity to the sequence in the lipA gene comprising:

contacting a sample with the polynucleotide of claim 1, wherein said polynucleotide hybridizes to said RNA, cDNA, or DNA when said RNA, cDNA, or DNA is present in the sample.

A more complete appreciation of the invention and many of the attendant advantages thereof will be readily obtained as the same becomes better understood by reference to the following detailed description.

### DETAILED DESCRIPTION OF THE INVENTION

As used herein, the term “L-amino acid” refers to the free amino acid or to a salt thereof. For example, whenever L-lysine or lysine is mentioned herein, this is intended also to include salts such as e.g. lysine monohydrochloride or lysine sulfate.

Polynucleotides containing sequences according to the invention are suitable as hybridization probes for RNA, cDNA and DNA in order to isolate nucleic acids and polynucleotides or genes of full length which code for lipoic acid synthetase or in order to isolate those nucleic acids and polynucleotides or genes which exhibit a high similarity to the sequence in the lipA gene. Furthermore, polynucleotides containing the sequences in accordance with the invention are suitable as primers with the help of which, using the polymerase chain reaction (PCR), DNA can be prepared from genes which code for lipoic acid synthetase.

Those oligonucleotides acting as probes or primers contain at least 30, preferably at least 20, very particularly preferably at least 15 nucleotides in sequence. Oligonucleotides with a length of at least 40 or 50 nucleotides are also suitable. Thus, such an oligonucletide may be 15 to 50 nucleotides in length. This range includes all specific values and subranges therebetween, such as 20, 25, 35 or 45 nucleotides.

The term “isolated” as used herein means separated from its natural surroundings. The term “polynucleotide” used herein generally refers to polyribonucleotides and polydeoxyribonucleotides, wherein this may be a non-modified RNA or DNA or a modified RNA or DNA.

Polynucleotides according to the invention include a polynucleotide in accordance with SEQ ID No. 1 or a fragment prepared therefrom and also those at least 70% of which, preferably at least 80% of which and in particular at least 90% to 95% of which is identical to the polynucleotide in accordance with SEQ ID No. 1 or a fragment prepared therefrom.

As used herein, the term “polypeptides” refers to peptides or proteins which contain two or more amino acids linked via peptide bonds.

Polypeptides according to the invention include a polypeptide in accordance with SEQ ID No. 2, in particular those with the biological activity of lipoic acid synthetase and also those at least 70% of which, preferably at least 80% of which and in particular at least 90% to 95% of which is identical to the polypeptide in accordance with SEQ ID No. 2 and have the activity described above.

Furthermore, the invention provides a process for the fermentative preparation of amino acids, in particular L-lysine, using coryneform bacteria which in particular already produce amino acids and in which the nucleotide sequences coding for the lipA gene are attenuated, in particular switched off or expressed at a low level.

The term “attenuation” as used herein describes the reduction in or switching off of the intracellular activity of one or more enzymes (proteins) in a microorganism which are coded by the corresponding DNAs by, for example, using a weak promoter or a gene or allele which codes for a corresponding enzyme with a low activity or by inactivating the corresponding gene or enzyme (protein) and, optionally, by combining these measures.

Microorganisms provided by the present invention can produce amino acids, in particular L-lysine, from glucose, saccharose, lactose, fructose, maltose, molasses, starch, cellulose or from glycerin and ethanol. They are representatives of coryneform bacteria, in particular of the genus Corynebacterium. From the genus Corynebacterium, the species Corynebacterium glutamicum which is recognized by persons skilled in the art for its ability to produce L-amino acids is mentioned in particular.

Suitable strains of the genus Corynebacterium, in particular of the species Corynebacterium glutamicum (C. glutamicum), are especially the known wild strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

or L-amino acid-producing mutants and strains prepared therefrom such as, for example, the L-lysine-producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum DM58-1

Corynebacterium glutamicum DG52-5

Corynebacterium glutamicum DSM 5715 and

Corynebacterium glutamicum DSM 12866

The inventors have succeeded in isolating, from C. glutamicum, the new lipA gene coding for lipoic acid synthetase.

In order to isolate the lipA gene, or also other genes, from C. glutamicum, a gene library from this microorganism is first prepared in Escherichia coli (E. coli). The preparation of gene libraries is described in generally known textbooks and manuals. As examples, the textbook by Winnacker: Gene und Klone, Eine Einfuhrung in die Gentechnologie (Verlag Chemie, Weinheim, Germany, 1990), or the manual by Sambrook et al.: Molecular Cloning, A Laboratory Manual (Cold Spring Harbor Laboratory Press, 1989) may be mentioned. A very well-known gene library is that of E. coli K-12 strain W3110, which was prepared by Kohara et al. (Cell 50, 495-508 (1987)) in λ vectors. Bathe et al. (Molecular and General Genetics, 252:255-265, 1996) describe a gene library from C. glutamicum ATCC13032, which was prepared by prepared with the aid of the cosmid vector SuperCos I (Wahl et al., 1987, Proceedings of the National Academy of Sciences USA, 84:2160-2164) in E. coli K-12 strain NM554 (Raleigh et al., 1988, Nucleic Acids Research 16:1563-1575). Bormann et al. (Molecular Microbiology 6(3), 317-326 (1992)) again describe a gene library from C. glutamicum ATCC13032 using the cosmid pHC79 (Hohn and Collins, 1980, Gene 11, 291-298).

To produce a gene library from C. glutamicum in E. coli plasmids such as pBR322 (Bolivar, 1979, Life Sciences, 25, 807-818) or pUC9 (Vieira et al., 1982, Gene, 19:259-268) may be used. Particularly suitable hosts are those E. coli strains which are restriction and recombination defective such as, for example strain DH5α (Jeffrey H. Miller: “A Short Course in Bacterial Genetics, A Laboratory Manual and Handbook for Escherichia coli and Related Bacteria”, Cold Spring Harbor Laboratory Press, 1992).

The long DNA fragments cloned with the aid of cosmids or other λ vectors may then be subdloned again in suitable vectors commonly used for DNA sequencing.

Methods for DNA sequencing are described, inter alia, in Sanger et al. (Proceedings of the National Academy of Sciences of the United States of America USA, 74:5463-5467, 1977).

The DNA sequences obtained may then be tested using well-known algorithms or sequence-analysis programs such as e.g. the program by Staden (Nucleic Acids Research 14, 217-232(1986)), the program by Marck (Nucleic Acids Research 16, 1829-1836 (1988)) or the GCG program by Butler (Methods of Biochemical Analysis 39, 74-97 (1998)).

The new DNA sequence from C. glutamicum coding for the lipA gene which, as SEQ ID No. 1, is a constituent of the present invention was obtained in this way. Furthermore, the amino acid sequence of the corresponding protein was derived from the available DNA sequence using the method described above. SEQ ID No. 2 represents the amino acid sequence of the resulting lipA gene product.

Coding DNA sequences which are produced from SEQ ID No. 1 due to degeneracy of the genetic code are also within the scope of the invention. In the same way, DNA sequences which hybridize with SEQ ID No. 1 or parts of SEQ ID No. 1 are within the scope of the invention. In the field of the invention, furthermore, conservative amino acid replacement such as e.g. replacement of glycine by alanine or of aspartic acid by glutamic acid in proteins, are known as “sense mutations” which do not lead to any fundamental change in the activity of the protein, i.e. they are functionally neutral. Furthermore, it is known that changes at the N- and/or C-terminus of a protein do not substantially impair, and may even stabilize, its function. A person skilled in the art can find information relating to this, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in well-known textbooks on genetics and molecular biology. Amino acid sequences which are produced in a corresponding way from SEQ ID No. 2 are also within the scope of the invention.

Finally, DNA sequences which are produced from SEQ ID No. 1 by the polymerase chain reaction (PCR) using primers are constituents of the invention. These types of oligonucleotides typically have a length of at least 15 nucleotides.

One skilled in the art can find instructions for identifying DNA sequences by means of hybridization, inter alia, in the manual “The DIG System Users Guide for Filter Hybridization” produced by Boehringer Mannheim GmbH (Mannheim, Germany, 1993) and in Liebl et al. (International Journal of Systematic Bacteriology 41:255-260 (1991)). Hybridization takes place under stringent conditions, that is to say the only hybrids formed are those in which probe and target sequence, i.e. the polynucleotides treated with the probe, are at least 70% identical. It is known that the stringency of hybridization, including the washing step, is affected or determined by varying the buffer composition, the temperature and the salt concentration. The hybridization reaction is preferably performed at relatively low stringency as compared with the washing step (Hybaid Hybridisation Guide, Hybaid Limited, Teddington, UK, 1996). A 5× SSC buffer at a temperature of about 50-68° C. can be used, for example, for the hybridization reaction. Here, probes also hybridize with polynucleotides which are less than 70% identical to the sequence of the probe. Such hybrids are less stable and are removed by washing under stringent conditions. This can be achieved, for example, by lowering the salt concentration to 2×SSC and optionally then to 0.5× SSC (The DIG System User's Guide for Filter Hybridization, Boehringer Mannheim, Mannheim, Germany, 1995), wherein the adjustments are carried out at a temperature of about 50-68° C. By the stepwise increase in the hybridization temperature from 50 to 68° C. in steps of about 1-2° C, polynucleotide fragments can be isolated which are, for example, at least 70% or at least 80% or at least 90% to 95% identical to the sequence in the probe used. Further instructions for hybridizing are obtainable on the market in the form of so-called kits (e.g. DIG Easy Hyb from Roche Diagnostics GmbH, Mannheim, Germany, Catalog No. 1603558).

One skilled in the art can find instructions for amplifying DNA sequences with the aid of the polymerase chain reaction (PCR), inter alia, in the manual by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

In the course of the work leading to the present invention, it was found that coryneform bacteria produce amino acids, in particular L-lysine, in an improved manner after attenuating the lipA gene.

To produce attenuation, either the expression of the lipA gene or the catalytic properties of the enzyme protein may be reduced or switched off. Optionally, both measures can be combined.

A reduction in gene expression can take place as a result of suitable culture management or by genetic modification (mutation) of the signal structures for gene expression. Signal structures for gene expression are, for example, repressor genes, activator genes, operators, promoters, attenuators, ribosome bonding positions, the start codon and terminators. A person skilled in the art can find information relating to these e.g. in patent application WO 96/15246, in Boyd and Murphy (Journal of Bacteriology 170:5949 (1988)), in Voskuil and Chambliss (Nucleic Acids Research 26:3548 (1998), in Jensen and Hammer (Biotechnology and Bioengineering 58:191 (1998)), in Patek et al. (Microbiology 142:1297 (1996)), Vasicova et al. (Journal of Bacteriology 181:6188 (1999)) and in well-known textbooks on genetics and molecular biology such as e.g. the textbook by Knippers (“Molekulare Genetik”, 6th edition, Georg Thieme Verlag, Stuttgart, Germany, 1995) or the textbook by Winnacker (“Gene und Klone”, VCH Verlagsgesellschaft, Weinheim, Germany, 1990).

Mutations which lead to a change or reduction in the catalytic properties of enzyme proteins are known in the field; the papers by Qiu and Goodman (Journal of Biological Chemistry 272:8611-8617 (1997)), Sugimoto et al. (Bioscience Biotechnology and Biochemistry 61:1760-1762 (1997)) and Möckel (“Die Threonindehydratase aus Corynebacterium glutamicum: Aufhebung der allosterischen Regulation und Struktur des Enzyms”, reports of the Jutlich Research Centre, Jül-2906, ISSN09442952, Julich, Germany, 1994) may be mentioned as examples. Reviews of the subject can be found in well-known textbooks on genetics and molecular biology such as e.g. the book by Hagemann (“Allgemeine Genetik”, Gustav Fischer Verlag, Stuttgart, 1986).

Suitable mutations are transitions, transversions, insertions and deletions. Depending on the effect of amino acid replacement on the enzyme activity, reference is made to missense mutations or nonsense mutations. Insertions or deletions of at least one base pair (bp) in a gene lead to frame shift mutations, as a result of which incorrect amino acids are incorporated or translation is terminated prematurely. Deletions of several codons lead typically to complete failure of enzyme activity. Instructions for producing these types of mutations are part of the prior art and can be found in well-known textbooks on genetics and molecular biology such as e.g. the textbook by Knippers (“Molekulare Genetik”, 6th edition, Georg Thieme Verlag, Stuttgart, Germany, 1995), the book by Winnacker (“Gene und Klone”, VCH Verlagsgesellschaft, Weinheim, Germany, 1990) or the book by Hagemann (“Allgemeine Genetik”, Gustav Fischer Verlag, Stuttgart, 1986).

A common method of mutating genes in C. glutamicum is the method of gene disruption and gene replacement described by Schwarzer and Piuhler (Bio/Technology 9,84-87 (1991)).

With the method of gene disruption a central part of the coding region of the gene being considered is cloned in a plasmid vector which can replicate in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jager et al., Journal of Bacteriology 174:5462-65 (1992)), pGEM-T (Promega corporation, Madison, Wis., USA), pCR2.1-TOPO (Shuman (1994). Journal of Biological Chemistry 269:32678-84; U.S. Pat. No. 5, 487,993), pCR®Blunt (Invitrogen, Groningen, Netherlands; Bernard et al., Journal of Molecular Biology, 234:534-541 (1993)) or pEMI (Schrumpf et al, 1991, Journal of Bacteriology 173:4510-4516). The plasmid vector which contains the central part of the coding region of the gene is then transferred by conjugation or transformation into the desired strain of C. glutamicum. The method of conjugation is described, for example, in Schäfer et al. (Applied and Environmental Microbiology 60, 756-759 (1994)). Methods for transforming are described, for example, in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene involved is disrupted by the vector sequence and two incomplete alleles are obtained, in which the 3′- or the 5′-ends respectively are each missing. This method was used, for example, by Fitzpatrick et al. (Applied Microbiology and Biotechnology 42, 575-580 (1994)) to switch off the recA gene in C. glutamicum.

With the method of gene replacement, a mutation such as e.g. a deletion, insertion or base replacement is produced in-vitro in the gene being considered. The allele produced is again cloned in a vector which does not replicate in C. glutamicum and this is then transferred by transformation or conjugation into the desired host for C. glutamicum. After homologous recombination by means of a first, integration-causing “cross-over” event and an appropriate second, excision-causing “cross-over” event in the target gene or in the target sequence, incorporation of the mutation or the allele is achieved. This method was used, for example, by Peters-Wendisch et al. (Microbiology 144, 915-927 (1998)) to switch off the pyc gene in C. glutamicum by means of a deletion.

A deletion, insertion or base replacement can be incorporated in the lipA gene in this way.

In addition, it may be advantageous for the production of L-amino acids, in particular L-lysine, in addition to attenuating the lipA gene in one or more enzymes on the relevant biosynthetic pathway, to enhance, in particular overexpress, glycolysis, anaploretic processes, the citric acid cycle, the pentose-phosphate cycle, amino acid export and optionally regulatory proteins.

Thus, for example, for the production of L-lysine, one or more endogeneous genes chosen from the group

the dapA gene coding for dihydrodipicolinate synthase (EP-B 0 197 335),

the gap gene coding for glyceraldehyde-3-phosphate dehydrogenase (Eikmanns (1992), Journal of Bacteriology 174:6076-6086),

the tpi gene coding for triosephosphate isomerase (Eikmanns (1992), Journal of Bacteriology 174:6076-6086),

the pgk gene coding for 3-phosphoglycerate kinase (Eikmanns (1992), Journal of Bacteriology 174:6076-6086),

the zwf gene coding for glucose-6-phosphate dehydrogenase (JP-A-09224661), the pyc gene coding for pyruvate carboxylase (DE-A-198 31 609), the lysC gene coding for a feed-back resistant aspartate kinase (Kalinowski et al. (1990), Molecular and General Genetics 224, 317-324; Accession No.P26512),

the mqo gene coding for malate-quinone-oxidoreductase (Molenaar et al., European Journal of Biochemistry 254, 395-403 (1998)),

the lysE gene coding for lysine export (DE-A- 195 48 222), or

the zwal coding for Zwal protein (DE: 199 59 328.0, DSM 13115) may be simultaneously enhanced, in particular overexpressed.

It may also be advantageous for the production of amino acids, in particular L-lysine, apart from attenuating the lipA gene, to simultaneously attenuate one or more genes chosen from the group

the pck gene coding for phosphoenolpyruvate carboxykinase (DE 199 50 409.1, DSM 13047),

the pgi gene coding for glucose-6-phosphate isomerase (US 09/396,478, DSM 12969),

the poxB gene coding for pyruvate oxidase (DE: 1995 1975.7, DSM 13114)

the zwa2 gene coding for Zwa2 protein (DE: 199 59 327.2, DSM 13113).

Furthermore, it may be advantageous for the production of amino acids, in particular L-lysine, apart from attenuating the lipA gene, to switch off undesired side reactions (Nakayama: “Breeding of Amino Acid Producing Microorganisms”, in: Overproduction of Microbial Products, Krumphanzl, Sikyta, Vanek (eds.), Academic Press, London, UK, 1982).

Microorganisms prepared according to the invention are also provided by the invention and may be cultivated continuously or batchwise in a batch process or in a fed batch process or repeated fed batch process for the purposes of producing L-amino acids, in particular L-lysine. A review of known cultivation processes is given in the text book by Chmiel (Bioprozesstechnik 1. Einflhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used has to comply in a suitable manner with the requirements of the particular strain. Descriptions of culture media for different microorganisms are given in the manual “Manual of Methods for General Bacteriology” by the American Society for Bacteriology (Washington D.C., USA, 1981). Sources of carbon which may be used are sugars and carbohydrates such as e.g. glucose, saccharose, lactose, fructose, maltose, molasses, starch and cellulose, oils and fats such as, for example, soya oil, sunflower oil, peanut oil and coconut oil, fatty acids such as, for example, palmitic acid, stearic acid and linoleic acid, alcohols such as, for example, glycerine and ethanol and organic acids such as, for example, acetic acid. These substances may be used individually or as a mixture.

Sources of nitrogen which may be used are organic nitrogen-containing compounds such as peptones, yeast extract, meat extract, malt extract, maize steep liquor, soya bean meal and urea or inorganic compounds such as ammonium sulfate, ammonium chloride, ammonium phosphate, ammonium carbonate and ammonium nitrate. The sources of nitrogen may be used individually or as a mixture.

Sources of phosphorus which may be used are phosphoric acid, potassium dihydrogen phosphate or dipotassium hydrogen phosphate or the corresponding sodium-containing salts. The culture medium preferably also contains salts of metals such as, for example, magnesium sulfate or iron sulfate, which are required for growth. Finally, essential growth-promoting substances such as amino acids and vitamins may be used in addition to the substances mentioned above. Suitable precursors may be added to the culture medium in addition to these. The feedstuffs mentioned may be added to the culture in the form of a single batch or be fed in a suitable manner during cultivation.

To regulate the pH of the culture, basic compounds such as sodium hydroxide, potassium hydroxide, ammonia or ammoniacal liquor or acid compounds such as phosphoric acid or sulfuric acid are used in an appropriate manner. To control the production of foam, antifoaming agents such as, for example, fatty acid polyglycol esters may be used. To maintain the stability of plasmids, suitable selectively-acting substances such as, for example, antibiotics, may be added to the medium. In order to maintain aerobic conditions, oxygen or oxygen-containing gas mixtures such as, for example, air, are passed into the culture. The temperature of the culture is normally 20° C. to 45° C. and is preferably 25° C. to 40° C. The culture procedure is continued until a maximum has been produced in the desired product. This objective is normally achieved within 10 hours to 160 hours.

Methods for determining L-amino acids are well-known. Analysis may be performed, for example, as described in Spackman et al. (Analytical Chemistry, 30, (1958), 1190) by anion exchange chromatography followed by ninhydrin derivation, or it may be performed by reversed phase HPLC as described in Lindroth et al. (Analytical Chemistry (1979) 51:1167-1174).

The invention also provides a process for the fermentative production of amino acids chosen from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophane and L-arginine, in particular L-lysine, using coryneform bacteria which in particular already produce one or more of the amino acids mentioned.

### EXAMPLES

The present invention is explained in more detail by means of the following examples. These examples are provided herein for purposes of illustration only and are not intended to be limiting unless otherwise specified.

The isolation of plasmid DNA from Escherichia coli and all techniques of restriction, Klenow treatment and alkaline phosphatase treatment were performed in accordance with Sambrook et al. (Molecular Cloning. A Laboratory Manual, 1989, Cold Spring Harbor Laboratory Press, Cold Spring Harbor, N.Y., USA). Methods for the transformation of Escherichia coli are also described in this manual. The composition of commonly used nutrient media such as LB or TY medium can also be found in therein.

### Example 1

### Production of a Genomic Cosmid Gene Library from C. glutarnicum ATCC 13032

Chromosomal DNA from C. glutamicum ATCC 13032 was isolated as described in Tauch et al., (1995, Plasmid 33:168-179), and partly cleaved with the restriction enzyme Sau3AI (Amersham Pharmacia, Freiburg, Germany, product description Sau3AI, Code no. 27-0913-02). The DNA fragments were dephosphorylated with shrimp alkaline phosphatase (Roche Molecular Biochemicals, Mannheim, Germany, product description SAP, Code no. 1758250). The DNA in the cosmid vector SuperCosl (Wahl et al. (1987), Proceedings of the National Academy of Sciences, USA 84:2160-2164), purchased from Stratagene (La Jolla, USA, product description SuperCosI Cosmid Vektor Kit, Code no. 251301) was cleaved with the restriction enzyme XbaI (Amersham Pharmacia, Freiburg, Germany, product description XbaI, Code no. 27-0948-02) and also dephosphorylated with shrimp alkaline phosphatase.

Then the cosmid DNA was cleaved with the restriction enzyme BamHI (Amersham Pharmacia, Freiburg, Germany, product description BamHI, Code no. 27-0868-04). The cosmid DNA treated in this way was mixed with the treated ATCC13032 DNA and the mixture was treated with T4 DNA ligase (Amersham Pharmacia, Freiburg, Germany, product description T4-DNA-Ligase, Code no. 27-0870-04). The ligation mixture was then packed into phages with the aid of Gigapack II XL Packing Extracts (Stratagene, La Jolla, USA, product description Gigapack II XL Packing Extract, Code no. 200217).

To infect E. coli strain NM554 (Raleigh et al. 1988, Nucleic Acid Res. 16:1563-1575), the cells were taken up in 10 mM MgSO₄ and mixed with an aliquot of the phage suspension. Infection and titering of the cosmid library were performed as described in Sambrook et al. (1989, Molecular Cloning: A Laboratory Manual, Cold Spring Harbor), wherein the cells were plated out on LB agar (Lennox, 1955, Virology, 1:190) +100 μg/ml ampicillin. After incubation overnight at 370° C., recombinant individual clones were selected.

### Example 2

### Isolating and Sequencing the lipA Gene

The cosmid DNA from an individual colony was isolated with the Qiaprep Spin Miniprep Kit (Product No. 27106, Qiagen, Hilden, Germany) in accordance with the manufacturer's information and partly cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Molecular Biochemicals, Mannheim, Germany, product description SAP, Product No. 1758250). After gel electrophoretic separation, isolation of the cosmid fragments in the size range 1500 to 2000 bp was performed with QiaExII Gel Extraction Kit (Product No. 20021, Qiagen, Hilden, Germany).

The DNA in sequencing vector pZero-1 purchased from Invitrogen (Groningen, Netherlands, product description Zero Background Cloning Kit, Product No. K2500-01) was cleaved with the restriction enzyme BamHI (Amersham Pharmacia, Freiburg, Germany, product description BamHI, Product No. 27-0868-04). Ligation of the cosmid fragments in sequencing vector pZero-1 was performed as described in Sambrook et al. (1989, Molecular Cloning: A Laboratory Manual, Cold Spring Harbor), wherein the DNA mixture was incubated overnight with T4 ligase (Pharmacia Biotech, Freiburg, Germany). This ligation mixture was then electroporated in E. coli strain DH5αCR (Grant, 1990, Proceedings of the National Academy of Sciences, U.S.A., 87:4645-4649) (Tauch et al. 1994, FEMS Microbiol Letters, 123:343-7) and plated out on LB agar (Lennox, 1955, Virology, 1:190) with 50 μg/ml zeocin.

The plasmid preparation of recombinant clones was performed with Biorobot 9600 (Product No. 900200, Qiagen, Hilden, Germany). Sequencing was performed using the dideoxy chain termination method according to Sanger et al. (1977, Proceedings of the National Academies of Sciences, U.S.A., 74:5463-5467) with modifications by Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. Gel electrophoretic separation and analysis of the sequencing reaction was performed in a “Rotiphorese NF Acrylamid/Bisacrylamid” Gel (29:1) (Product No. A124.1, Roth, Karlsruhe, Germany) using the “ABI Prism 377” sequencing instrument from PE Applied Biosystems (Weiterstadt, Germany).

The crude sequencing data obtained were then processed using the Staden program package (1986, Nucleic Acids Research, 14:217-231) Version 97-0. The individual sequences of the pZero 1 derivatives were assembled to give a cohesive contig. Computer aided coding region analyses were drawn up with the program XNIP (Staden, 1986, Nucleic Acids Research, 14:217-231). Further analyses were performed using the “BLAST search programs” (Altschul et al., 1997, Nucleic Acids Research, 25:33893402) against the non-redundant database of the “National Center for Biotechnology Information” (NCBI, Bethesda, Md., USA).

The nucleotide sequence obtained is shown in SEQ ID No. 1. Analysis of the nucleotide sequence produced an open reading frame of 1047 bp, which was called the lipA gene. The lipA gene coded for a polypeptide of 348 amino acids, which is shown in SEQ ID NO. 2.

Obviously, numerous modifications and variations of the present invention are possible in light of the above teachings. It is therefore to be understood that within the scope of the appended claims, the invention may be practiced otherwise than as specifically described herein.

All of the articles and publications cited above are incorporated herein by reference.

This Application is based on German Patent Application Ser. No. 100 42 742.1, filed on Aug. 31, 2000, and incorporated herein by reference.

1. A polynucleotide isolated from coryneform bacteria, containing a polynucleotide sequence coding for the lipA gene selected from the group consisting of:
(a) a polynucleotide which is at least 70% identical to a polynucleotide which codes for a polypeptide which contains the amino acid sequence of SEQ ID No. 2,
(b) a polynucleotide which codes for a polypeptide which contains an amino acid sequence which is at least 70% identical to the amino acid sequence of SEQ ID No. 2,
(c) a polynucleotide which is complementary to the polynucleotide (a) or (b), and
(d) a polynucleotide containing a sequence of at least 15 nucleotides from the polynucleotide (a), (b), or (c).

2. The polynucleotide of claim 1, wherein the polypeptide has the activity of lipoic acid synthetase.

3. The polynucleotide of claim 1, wherein the polynucleotide is recombinant DNA which can replicate in coryneform bacteria.

4. The polynucleotide of claim 1, which is a RNA.

5. The polynucleotide of claim 3, which contains the nucleic acid sequence shown in SEQ ID No. 1.

6. The polynucleotide of claim 3, which contains
(i) the nucleotide sequence shown in SEQ ID No. 1, or
(ii) at least one nucleotide sequence which corresponds to sequence (i) within the region of degeneration of the genetic code, or
(iii) at least one nucleotide sequence which hybridizes with the sequences complementary to nucleotide sequence (i) or (ii), and, optionally,
(iv) functionally neutral sense mutations in nucleotide sequence (i).

7. The polynucleotide of claim 6, wherein the nucleotide sequence (iii) hybridizes under a stringency corresponding to at most 2× SSC.

8. The polynucleotide of claim 3, which codes for a polypeptide which contains the amino acid sequence shown in SEQ ID No. 2.

9. Coryneform bacteria in which the lipA gene is attenuated.

10. The bacteria of claim 9, in which the lipA gene is switched off.

11. A process for preparing an L-amino acid, comprising:
(a) fermenting a coryneform bacteria which produces the L-amino acid and in which at least the lipA gene is attenuated,
(b) enriching the L-amino acid in the medium or in the cells of the bacteria, and
(c) isolating the L-amino acid.

12. The process of claim 11, wherein the amino acid is L-lysine.

13. The process of claim 11, wherein genes in the biosynthetic pathway of the L-amino acid are enhanced in the coryneform bacteria.

14. The process of claim 11, wherein at least some of the metabolic pathways which reduce the formation of the L-amino acid are switched off in the coryneform bacteria.

15. The process of claim 11, wherein the expression of the polynucleotide(s) which codes (code) for the lipA gene is reduced.

16. The process of claim 11, wherein the expression of the polynucleotide(s) which codes (code) for the lipA gene is switched off.

17. A process of claim 11, wherein the catalytic properties of the polypeptide for which the polynucleotide lipA codes are reduced.

18. A process of claim 11, wherein one or more of the genes selected from the group consisting of
the endogenous dapA gene coding for dihydrodipicolinate synthase,
the endogenous gap gene coding for glyceraldehyde-3-phosphate dehydrogenase,
the endogenous tpi gene coding for triosephosphate isomerase,
the endogenous pgk gene coding for 3-phosphoglycerate kinase,
the endogenous zwf gene coding for glucose-6-phosphate dehydrogenase gene,
the endogenous pyc gene coding for pyruvate carboxylase,
the endogenous lysC gene coding for a feed-back resistant aspartate kinase,
the endogenous mqo gene coding for malate-quinone-oxidoreductase,
the endogenous lysE gene coding for lysine export, and
the endogenous zwal gene coding for Zwal protein
are simultaneously enhanced in the coryneform bacteria.

19. The process of claim 18, wherein said one or more genes are overexpressed.

20. A process of claim 11, wherein one or more genes selected from the group consisting of
the pck gene coding for phosphoenolpyruvate carboxykinase,
the pgi gene coding for glucose-6-phosphate isomerase,
the poxB gene coding for pyruvate oxidase, and
the zwa2 gene coding for Zwa2 protein
are simultaneously attenuated.

21. The process of claim 11, wherein the microorganisms is of the genus Corynebacterium.

22. A process for identifying RNA, CDNA, or DNA which code for lipoic acid synthetase or exhibit a high similarity to the sequence in the lipA gene comprising:
contacting a sample with the polynucleotide of claim 1, wherein said polynucleotide hybridizes to said RNA, cDNA, or DNA when said RNA, CDNA, or DNA is present in the sample.

23. The process of claim 22, further comprising isolating said RNA, cDNA, or DNA.


SEQUENCE LISTING (2 sequences)

SEQ ID NO: 1 - DNA, 1539 bp, Corynebacterium glutamicum
  CDS (345)..(1388)
tttctatgagcacatcattccgtgtggcattgctgatgcaggcttgagcacactctcgagggaactgaaaagggacgtttcagttgaggaattagtcgagccatcgatccgcgcattggatgatgctttggctggtcggctggttgtttctgatcattctttcggcagcgcgcccgacccaactaagaatctccctaaacgggggtagtacgaggaattttgtcggtggggcgcctcgttgaagcgaagtagagccgattgcagaatcggcggaatgagacgtcgaaaagcgtttaagctttccctaaaaatatcactaactcgaaagatgtaaggttgcatttgtgactatcgcacctgaaggacgacgactgctacgcgtcgaagctcgaaactcagaaaccccgattgagacgaagcctcgatggattagaaaccaggtcaaaaacggacctgagtatcaggatatgaaggaacgtgtcgctggcgcatcactacacactgtgtgtcaggaggctggctgtcctaatatccatgagtgttgggaatcccgtgaggcaaccttcctcattggtggcgccaactgctctcgccgctgtgatttctgcatgatcaactcggctcgccctgagccactcgaccgcggtgagccactgcgtgtcgctgagtctgttcgtgagatgcagctgaattactccaccatcaccggtgttacccgtgatgatctggatgatgaaggcgcatggctgtactcagaagtggttcgtaagatccacgagctgaacccacacaccggtgtggaaaacctggtgcctgatttctccggcaagaaggatctgctgcaggaagtttttgaatcccgcccagaggttttcgctcacaacgtggaaactgtgccacgtattttcaagcgcattcgcccagcattccgctacgagcgttcacttgatgtgatccgtcaggctcgcgatttcggtctggtgaccaagtccaacctgattttgggcatgggtgaaaccaaggaagaaatcaccgaggcgctgcaggatctgcacgacgctggctgtgacatcatcaccatcacccagtacctgcgtcctggtcctttgttccaccccatcgagcgttgggtgaagcctgaggagttcctcgagcacgctgatgctgcaaaggaaatgggcttcgctgctgttatgtccggcccattggttcgttcctcttaccgtgcaggccgtctgtacgcgcaggccatggagttccgtggcgaggaaatcccagcacacctcgcgcacctgaaggatacttccggaggatccaccgcccaggaagcatctacacttctggagcgttacggtgcttccgaagacaccccagtggtgtccttcaactaagcccgaagttttttaaccgccgcattcgatcaccaaatgtggcggttttgcgtcgaaaagcgtgctctttctacacctctttgaggttcattttcgcggtttcctcacaatcgcctattgttaagtacatggcagacgcgaaaaagca

SEQ ID NO: 2 - protein, 348 aa, Corynebacterium glutamicum
MTIAPEGRRLLRVEARNSETPIETKPRWIRNQVKNGPEYQDMKERVAGASLHTVCQEAGCPNIHECWESREATFLIGGANCSRRCDFCMINSARPEPLDRGEPLRVAESVREMQLNYSTITGVTRDDLDDEGAWLYSEVVRKIHELNPHTGVENLVPDFSGKKDLLQEVFESRPEVFAHNVETVPRIFKRIRPAFRYERSLDVIRQARDFGLVTKSNLILGMGETKEEITEALQDLHDAGCDIITITQYLRPGPLFHPIERWVKPEEFLEHADAAKEMGFAAVMSGPLVRSSYRAGRLYAQAMEFRGEEIPAHLAHLKDTSGGSTAQEASTLLERYGASEDTPVVSFN